(19) United States
(12) Patent Application Publication
(10) Pub. No.: US 2008/0194986 A1
(43) Pub. Date: Aug. 14, 2008

(54) BLOOD COLLECTION SET WITH VENTING MECHANISM

(75) Inventors: Hugh Conway, Verona, NJ (US); Hareesh Nair, Milton, MA (US); Kirk Swenson, West Caldwell, NJ (US); C. Mark Newby, Tuxedo, NY (US); Curtis Bloch, Sandy, UT (US); Richard Levy, Sumter, SC (US); Bradley Wilkinson, North Haledon, NJ (US); James Schneider, Wayne, NJ (US); Bryan Towns, Union Bridge, MD (US); Michael Iskra, Bridgewater, NJ (US); Stewart Marsden, Montville, NJ (US); Robert G. Ellis, Mahwah, NJ (US)

Correspondence Address: David W. Highet, VP & Chief IP Counsel;Becton, Dickinson and Company, 1 Becton Drive, MC 110, Franklin Lakes, NJ, 07417-1880, US

(73) Assignee: BECTON, DICKINSON AND COMPANY, Franklin Lakes, NJ (US)

(21) Appl. No.: 11/971,679

(22) Filed: Jan. 9, 2008

Related U.S. Application Data
(63) Continuation of application No. 11/141,446, filed on May 31, 2005.
(60) Provisional application No. 60/576,140, filed on Jun. 2, 2004.

(51) Int. Cl. A61B 5/15

(52) U.S. Cl. 600/579; 600/576

(57) ABSTRACT

The blood collection set includes an IV needle assembly, a length of flexible plastic tubing extending from the IV needle assembly and a non-patient needle assembly. The set is formed with a venting mechanism that permits an outflow of air, while blocking an outflow of blood or other fluids. Thus, the venting mechanism enables air that had existed in interior portions of the blood collection set to be vented allowing for greater flash visualization on venous entry, and avoids the need to employ a discard tube.

The present application is a continuation of U.S. patent application Ser. No. 11/141,446, filed May 31, 2005, which claims priority to U.S. Provisional Patent Application Ser. No. 60/576,140, which was filed on Jun. 2, 2004.

### BACKGROUND OF THE INVENTION

1. Field of the Invention

The subject invention relates to a blood collection set with self-venting features.

2. Description of the Related Art

Phlebotomy procedures often are carried out using a blood collection set. A typical blood collection set includes an IV needle assembly with an IV cannula that has a proximal end, a sharply pointed distal end and a lumen extending between the ends. The needle assembly also includes a plastic IV hub with a proximal end, a distal end, and a passage extending between the ends. The proximal end of the IV cannula is mounted in the passage of the IV hub so that the lumen through the IV cannula communicates with the passage through the IV hub. The needle assembly may further include a shield for shielding the IV cannula after use and a packaging cover for safely covering the IV cannula prior to use. Packaging covers typically are rigid tubes with a proximal end that can be telescoped over the IV cannula and frictionally engaged with the distal end of the IV hub. Shields for blood collection sets have taken many forms. Some shields are telescoped over the IV hub and can be moved from a proximal position where the cannula is exposed to a distal position where the cannula is shielded. Other shields are hinged to the IV hub and can be rotated from an open position where the IV cannula is exposed to a closed position where the IV cannula is shielded. A needle assembly for a blood collection set also may include two flexible wings that project transversely from the IV hub or from the shield. The wings can be folded into face-to-face relationship with one another to effectively define a handle that facilitates manipulation of the needle assembly. The wings then can be rotated away from one another and held against the skin of the patient.

Blood collection sets also include a length of flexible plastic tubing. The tubing has a distal end that is connected to the proximal end of the IV hub. The tubing also has a proximal end that is connected to a plastic fitting. Thus, fluid communication is provided between the lumen of the IV cannula and the plastic fitting at the proximal end of the flexible tubing. The plastic fitting may be a female luer fitting that can be connected to a male luer fitting. The fitting then can be placed in communication with a reservoir or container for collecting a sample of blood.

Phlebotomy procedures often employ evacuated tubes, such as the VACUTAINER® brand of evacuated tubes sold by Becton Dickinson and Company. Evacuated tubes often are used with a tube holder that has a proximal end, a distal end, and a tubular side wall extending between the ends. The proximal end of the holder is widely open and is configured for slidably receiving the evacuated tube. The distal end of the holder typically includes an end wall with a mounting aperture. The mounting aperture includes internal threads or other mounting structures.

The tube holder may be used with a non-patient needle assembly that has a non-patient hub with external surface configurations for mounting in the mounting aperture of the holder. The non-patient needle assembly further includes a non-patient cannula extending proximally from the hub and a multiple sample sleeve telescoped over the non-patient cannula and mounted to the proximal end of the hub. The hub of the non-patient needle assembly can be threaded or otherwise engaged in the mounting aperture of the tube holder so that the non-patient needle and the multiple sample sleeve project into the tube receiving chamber of the holder.

The blood collection set may be used by mounting the fitting at the proximal end of the flexible plastic tubing to the distal end of the hub of the non-patient needle assembly. The packaging shield that covers the non-patient cannula then may be removed, and the hub of the non-patient needle assembly may be engaged with the tube holder. The medical practitioner then grips the IV needle assembly and removes the packaging cover from the IV cannula. The gripping of the IV needle assembly may include folding the flexible wings into face-to-face engagement and gripping the folded wings between a thumb and forefinger. The pointed distal end of the IV cannula then is urged into a targeted blood vessel. The wings then may be folded into engagement with the skin of the patient and may be taped in position. An evacuated tube then is urged into the open proximal end of the blood collection tube holder so that the non-patient needle pierces the stopper of the evacuated tube. As a result, the blood vessel of the patient is placed in communication with the interior of the evacuated tube, and the pressure differential between the blood vessel and the evacuated tube will generate a flow of blood through the IV cannula, through the passage of the IV hub, through the flexible tubing, through the non-patient hub and finally through the non-patient needle and into the evacuated tube.

It will be appreciated that a significant volume of air must be displaced before blood enters the evacuated tube. This air will be displaced by the flowing blood and will be urged into the evacuated tube. The flow of air into the evacuated tube increases the air pressure in the tube and offsets the pressure differential that generates the flow of blood from the patient to the evacuated tube. Thus, blood flow is slowed. Blood flow into the blood collection tube may stop when the pressure in the tube equals the fluid pressure of the blood. In effect air from the blood collection set reduces the volume of blood collected into the tube. The reduced blood volume can be undesirable such as when it adversely affects the ratio of additive to blood within the tube. An example is when the tube contains the additive citrate for clotting time studies in which the ratio of blood to citrate is critical.

Medical practitioners have several approaches for addressing problems relating to air in a blood collection set at the start of a phlebotomy procedure. For example, the first tube of collected blood may be considered a discard tube. Thus, the evacuated tube will remain in communication with the non-patient needle until blood begins to flow into the tube. The tube then will be removed and discarded and a second tube will be inserted into the holder for collecting a sample that can be used reliably. This approach adds to the cost and time of the procedure and wastes blood. Some medical practitioners try to vent air from the system before the first blood collection tube is placed in communication with the non-patient needle. This approach also wastes blood and can lead to contamination or accidental sticks depending upon the method of venting.

The typical needle hub is formed from an opaque plastic material, and plastic tubing often is formed from a translucent plastic material. Neither the opaque plastic material nor the translucent flexible tubing provide a clear indication of venous or arterial access. Blood flow into an evacuated tube does provide an indication of venous or arterial access. However, the initial movement of air into the evacuated tube is delayed until the evacuated tube is added onto the non patient needle. Thus, a medical practitioner may have a delayed indication of venous or arterial access and may incorrectly assume that the blood vessel was not accessed properly. In these situations, the medical practitioner may try to access the blood vessel again even though the initial access was successful. Accordingly, the patient may be subjected to unnecessary trauma during a repeated attempt to access the targeted blood vessel. Thus, improved techniques for dealing with the issue of air trapped in tubing would be desirable.

### SUMMARY OF THE INVENTION

The invention is a self-venting blood collection set with a self-venting mechanism that permits escape of air during use, and which, typically, also prevents an outflow of fluid, such as blood. As used herein, venting mechanism indicates one or more features or elements that provide venting of air, but which, typically, prevent fluid from passing through. Thus, air under venous pressure will be allowed to escape from the blood collection set through the mechanism until blood reaches the venting mechanism. The venting mechanism then will seal, or prevent blood flow through or around it, to prevent blood leakage and allow blood to be collected into evacuated collection tubes or into other appropriate blood collection receptacles. The invention thus provides good flash visualization, as well as the capability to provide a blood collection set that does not require a discard tube, without affecting accepted blood collection processes. A variety of venting mechanisms, venting media and venting locations are suitable, as set forth below. (As used herein, venting mechanism indicates the combination of elements, configurations, materials, etc. that provide the venting. As used herein, venting media indicates the actual element that vents the air, e.g., plug, coating, finish, etc.)

The blood collection set preferably includes an IV needle assembly, a length of flexible plastic tubing extending from the IV needle assembly and a non-patient needle assembly. The venting mechanism preferably is disposed on or near the non-patient needle assembly to permit venting of a maximum amount of the air that is in the blood collection set prior to the initiation evacuated tube use.

The IV needle assembly typically comprises an IV hub having a proximal end, a distal end and a passage extending between the ends. The IV needle assembly typically comprises an IV cannula having a proximal end mounted in the passage of the IV hub, a pointed distal end projecting distally from the IV hub and a lumen that communicates with the passage through the IV hub. The flexible tubing is connected to the proximal end of the IV hub. The IV needle assembly typically includes a packaging cover that protectively encloses the IV needle cannula prior to use. The packaging cover is removed immediately prior to use to permit access to the IV cannula. The IV needle assembly may further include a protective shield that is moveable relative to the IV cannula from an open position where the IV cannula is exposed to a closed position where the IV cannula is substantially shielded. The shield protects against accidental sticks with the used IV cannula. A pair of flexible wings may be mounted to the IV hub or to the shield to facilitate manipulation of the IV needle assembly.

The non-patient needle assembly includes a non-patient hub having a proximal end and a distal end. The non-patient needle assembly further includes a non-patient cannula having a distal end securely mounted in the hub, a proximal end projecting proximally from the non-patient hub and a lumen that communicates with the passage through the non-patient hub. A multiple sample sleeve is typically mounted over the non-patient cannula and secured to the proximal end of the non-patient hub. External portions of the non-patient hub near the proximal end thereof may be formed with an array of external threads or other mounting structure to enable the non-patient needle assembly to be mounted to a collection tube holder or other such medical device. Or, the holder may be pre-attached with the non-patient needle assembly. The blood collection set may further include a fitting mounted to the proximal end of the flexible plastic tubing and configured for mating with the distal end of the non-patient hub. For example, the fitting may be a female luer fitting that can be engaged with the male luer taper at the distal end of the non-patient hub.

In one embodiment, the venting mechanism is located at or near the non-patient hub, e.g., in the hub itself near the distal end of the non-patient needle or in the tubing itself at a proximal end thereof. The venting mechanism thereby provides communication between the passage and the surrounding environment either through the passage itself or through the non-patient hub. Alternatively, wherein the venting mechanism is located in the tubing, any location along the tubing is possible.

In a further embodiment, the venting mechanism location is in a space between a female and male luer interface or within a female luer and therefore will be at or near to the non-patient needle so that only a small amount of air will be collected with the first sample of blood.

In another embodiment, the venting mechanism is located beyond the non-patient cannula proximal end, which means that the air passes through the non-patient cannula proximal end from which blood is drawn, and then through the vent. Specifically, air is vented from the fluid passage and out of the non-patient cannula proximal end where it further flows through the space between needle exterior and multiple sample sleeve. The air then flows through the venting mechanism, which may be at the non-patient barb, the non-patient hub thread, the non-patient hub body, the multiple sample sleeve, or other location or combination of locations that are beyond the non-patient cannula proximal end. The collection tube, which is applied at the non-patient cannula proximal end, draws blood from only the fluid passage and not from the vent space. This embodiment thus enables blood to flow through the entire collection path for full tubing flash, eliminating the need for a discard tube and maintaining the desired blood to additive ratio. It also avoids the blood specimen coming in contact with the vent, which could potentially cause platelet activation, contamination or other undesirable result. It also avoids air being sucked back into the fluid passage when the evacuated tube is applied.

In a further embodiment, the venting mechanism is an opening in the side of the non-patient cannula, combined with a vent media, which vents air but not blood, e.g. a vent plug consisting of a hydrophobic material.

Another embodiment of this invention has a venting mechanism comprised of a unified non-patient hub that is at least partially constructed of porous material such as sintered plastic, ceramic or metal. The porous material can be arranged to provide venting of air either before that air enters the non-patient cannula, or after the air flows through the cannula, out the proximal end, and into the space between the cannula and a multiple sample sleeve. The porous material provides venting of the air but blocks leakage of the blood. In the typical embodiment, the porous material is hydrophobic. The porous material may further contain or be coated with materials that swell upon wetting to further contain the blood. Other venting methods are also possible. The internal passage wall's surface may be coated with a sealant to prevent contamination of the blood sample by the porous material. This embodiment enables blood to flow through the entire collection path for full tubing flash, and also enables elimination of the waste tube or variability in blood to additive ratio. Optionally, the hub can be permanently bonded to a tube holder obviating the need or inconvenience of threaded connections. Bonding to the holder may be accomplished by solvent, welding, heat, pressure or and other convenient means or combination thereof. Such an integrated device is highly efficient to manufacture, and promotes safe medical practice by having the holder be discarded with the needle.

In a further embodiment, the venting mechanism involves venting air through a side opening located somewhere along the fluid passage to the exterior, where the opening is covered by a venting material having a shape which mechanically holds the venting material in or on the opening. Preferably, the vent material is hydrophobic such that the surface tension also prevents leakage. The vent media material in this embodiment typically has an elastic property and shape such that spring energy holds the vent material onto the device. For example, it is possible to use a C-shaped vent in which distortion of the shape is required for the vent to stretch over the receiving structure on the hub. Once the vent is placed over the receiving structure, it is released and fully maintained in place using it's own resiliency and in absence of bonding materials such as epoxies, which could be disadvantageously absorbed into the vent. The vent mechanism of this embodiment could alternatively involve first compressing a vent material, placing the material into the opening, and releasing the vent material to expand into the opening. This embodiment enables efficient mass production.

In a further embodiment, the venting mechanism utilizes a branch in the fluid passage, e.g., a “Y” or “T”. The branching may be at any location or locations along the fluid passage, but is preferably at the proximal end such as at the non-patient hub. The branching may be in the form of a separate component added into the fluid passage such as in between the female and male luer fittings or it may be integral within the hub. The branching includes some type of vent media, as discussed herein.

### BRIEF DESCRIPTION OF THE DRAWINGS

FIG. 1A is a perspective view of a blood collection set and collection tube holder.

FIG. 1B is a top plan view of the blood collection set and collection tube holder shown in FIG. 1.

FIG. 2 is side elevation view of the non-patient needle assembly, partly in section.

FIG. 3 is a cross-sectional side view of an embodiment of the female luer in the non-patient needle assembly of the blood collection set.

FIG. 4 is also a cross-sectional side view similar to FIG. 3, but showing an alternate embodiment of the invention.

FIG. 5 is a side elevation view of FIG. 4 from the aspect of Z-Z.

FIG. 6 is also a cross-sectional side view similar to FIG. 3, but showing an alternate embodiment of the invention.

FIG. 7 is a side elevation view of FIG. 6.

FIG. 8 is a cross-sectional side view of the female to male luer interface in this embodiment of the non-patient needle assembly of the blood collection set.

FIG. 9 is a perspective view of an embodiment of a female luer design in the non-patient needle assembly of the blood collection set.

FIG. 10 is a perspective view similar to FIG. 9, but showing an alternate embodiment of the invention.

FIG. 11 is a perspective view similar to FIG. 9, but showing an alternate embodiment of the invention.

FIG. 12 is a perspective view similar to FIG. 9, but showing an alternate embodiment of the invention.

FIG. 13 is a cross-sectional side view of the female to male luer interface in this embodiment of the non-patient needle assembly of the blood collection set.

FIG. 14 is a cross-sectional side view of an embodiment of the male luer non-patient assembly

FIG. 15A is also a cross-sectional side view similar to FIG. 14, but showing an alternate embodiment of the invention.

FIG. 15B is also a cross-sectional side view similar to FIG. 14, but showing an alternate embodiment of the invention.

FIG. 15C is a magnified view of FIG. 15B from the aspect of Detail B.

FIG. 16A is also a cross-sectional side view similar to FIG. 14, but showing an alternate embodiment of the invention.

FIG. 16B is a side elevation view of FIG. 16A from the aspect of X-X.

FIG. 17A is also a cross-sectional side view similar to FIG. 14, but showing an alternate embodiment of the invention.

FIG. 17B is a side elevation view of FIG. 17A from the aspect of Y-Y.

FIG. 18 is also a cross-sectional side view similar to FIG. 14, but showing an alternate embodiment of the invention.

FIG. 19 is also a cross-sectional side view similar to FIG. 14, but showing an alternate embodiment of the invention.

FIG. 20 is also a cross-sectional side view similar to FIG. 14, but showing an alternate embodiment of the invention.

FIG. 21 is a cross-sectional side view of an embodiment of the interface between the non-patient cannula hub and the male luer hub.

FIG. 22 is a cross-sectional side view of an embodiment of the non-patient needle assembly.

FIG. 23 is a cross-sectional side view of an embodiment of the non-patient needle assembly of the blood collection set.

FIG. 24 is also a cross-sectional side view similar to FIG. 23, but showing an alternate embodiment of the invention.

FIG. 25A is a cross-sectional side view of an embodiment of the non-patient needle assembly of the blood collection set.

FIG. 25B is a side elevation view of FIG. 25A from the aspect of R-R.

FIG. 26 is a perspective view of an embodiment of the female and male luer interface in the non-patient needle assembly of the blood collection set.

FIG. 27 is a perspective view of an embodiment of the non-patient barb multiple sample sleeve interface in the non-patient needle assembly of the blood collection set.

FIG. 28 is a cross-sectional view of an embodiment of the breathable cord design.

FIG. 29 is also a cross-sectional side view similar to FIG. 28, but showing an alternate embodiment of the invention.

FIG. 30 is also a cross-sectional side view similar to FIG. 14, but showing an alternate embodiment of the invention.

FIG. 31 is also a cross-sectional side view similar to FIG. 14, but showing an alternate embodiment of the invention.

FIG. 32 is also a cross-sectional side view similar to FIG. 14, but showing an alternate embodiment of the invention.

FIG. 33 is also a cross-sectional side view similar to FIG. 14, but showing an alternate embodiment of the invention.

FIG. 35 is also a cross-sectional side view similar to FIG. 14, but showing an alternate embodiment of the invention.

FIG. 36 is also a cross-sectional side view similar to FIG. 14, but showing an alternate embodiment of the invention.

FIG. 37 is also a cross-sectional side view similar to FIG. 14, but showing an alternate embodiment of the invention.

FIG. 38 is a cross-sectional side view of an embodiment of the non-patient needle assembly of the blood collection set.

FIG. 39 is a cross-sectional side view of the flexible tubing in an embodiment of the blood collection set.

FIG. 40 is a perspective view of one embodiment of the blood collection set.

FIG. 41 is a perspective view similar to FIG. 40, but showing an alternate embodiment of the invention.

FIG. 42 is a cross-sectional side view of the non-patient needle assembly of the blood collection set.

### DETAILED DESCRIPTION

The invention is a self-venting blood collection set with a self-venting mechanism that permits escape of air during use which, typically, also prevents an outflow of fluid, such as blood. As used herein, venting mechanism indicates one or more features or elements that provide venting of air, but which, typically, prevent fluid from passing through.

It should be noted that the vent media could be, for example, a distinct physical element such as a plug or insert, a integral portion of a device that has been treated such as by laser drilling or has been formed in whole or in part from a porous material, or a coating, layer, etc. formed by disposing a material onto the device, e.g., by dipping, coating, spraying or the like.

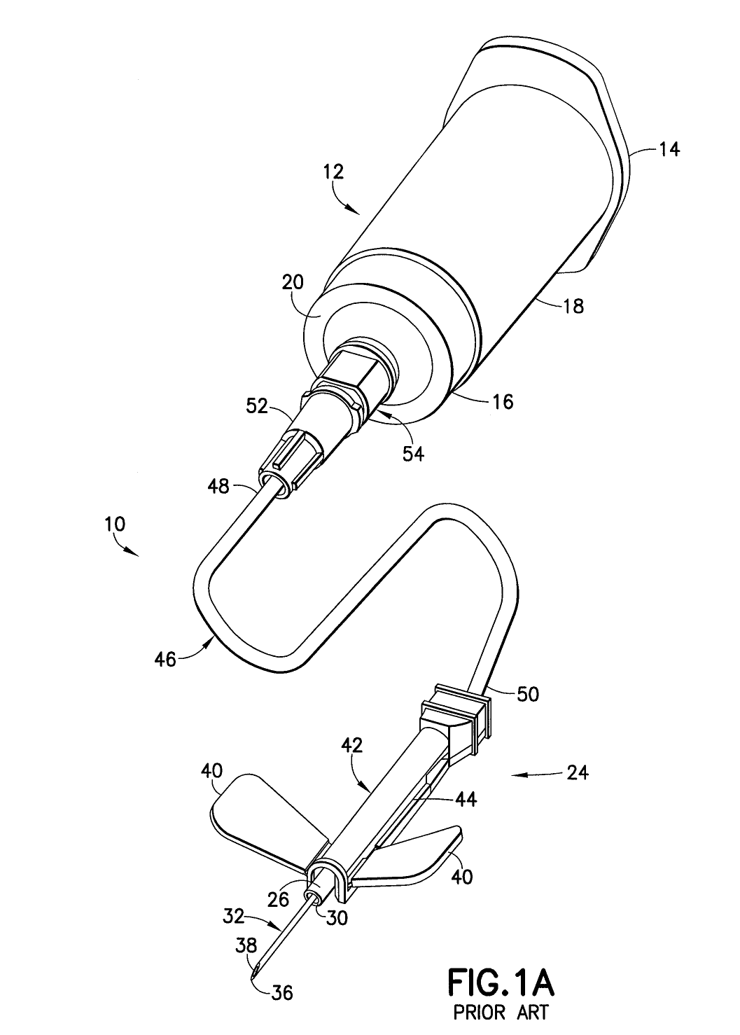

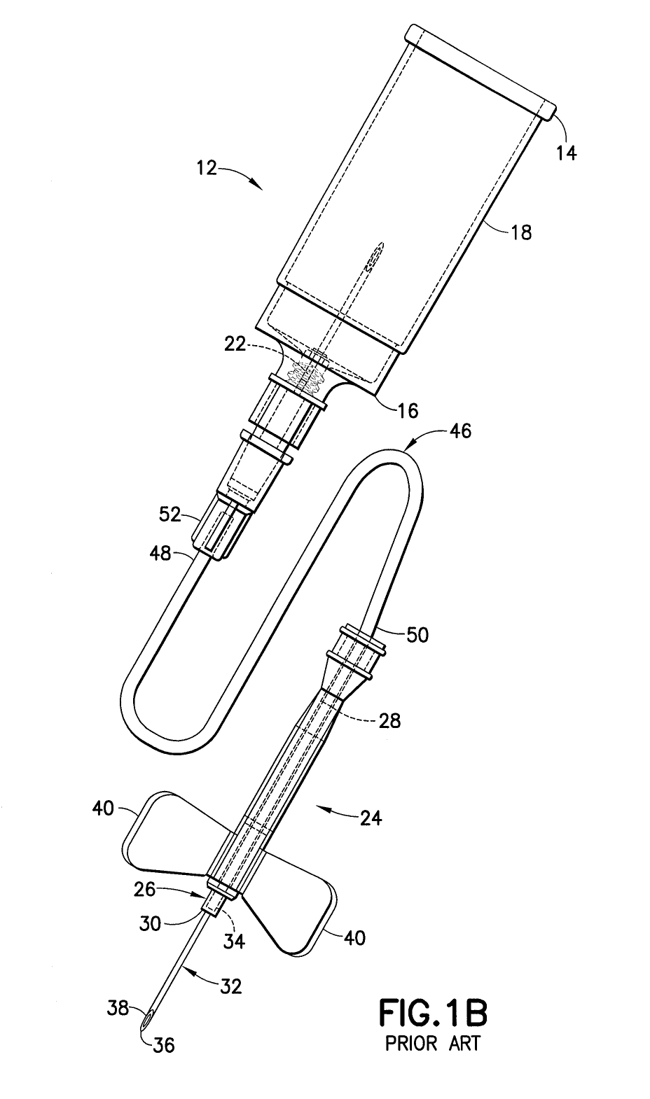

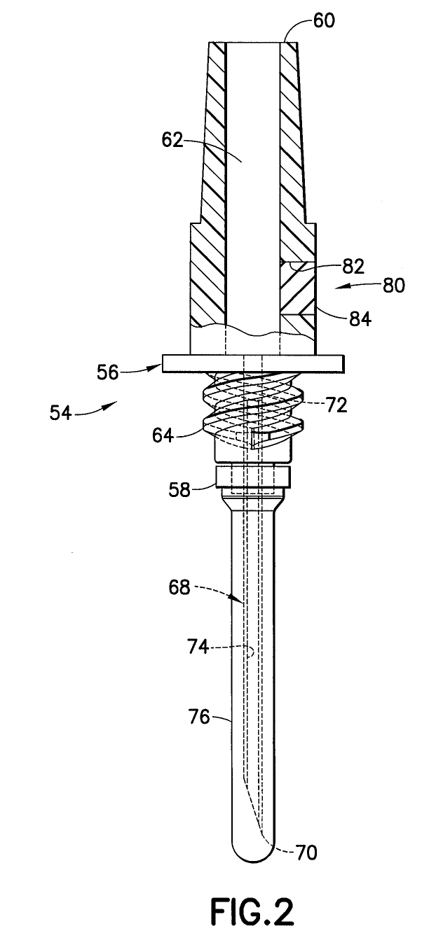

A prior art blood collection set in accordance with the subject invention is identified generally by the numeral 10 in FIGS. 1A and 1B. Blood collection set 10 is employed in this embodiment with a collection tube holder 12. Holder 12 has a proximal end 14, a distal end 16 and a tubular sidewall 18 extending between the ends. Proximal end 14 of holder 12 is widely open and defines an entry to a tube receptacle within sidewall 18. Thus, an evacuated collection tube can be slid in a proximal-to-distal direction through open proximal end 14 of holder 12 toward distal end 16. Distal end 16 of holder 12 is characterized by an end wall 20. End wall 20 is formed with an internally threaded mounting aperture 22, as shown in FIG. 2.

Blood collection set 10 includes an IV needle assembly 24 that comprises an IV hub 26. IV hub 26 includes a proximal end 28, a distal end 30 and a passage (not shown) extending between the ends. IV needle assembly 24 further includes an IV cannula 32 with a proximal end 34, a pointed distal end 36 and a lumen 38 extending between the ends. Proximal end 34 of IV cannula 32 is mounted securely in the passage of IV hub 26. Thus, lumen 38 through IV cannula 32 communicates with the passage through IV hub 26. Flexible wings 40 are mounted to IV hub 26 at a location near distal end 30. Wings 40 can be folded into face-to-face relationship with one another for convenient gripping between a thumb and forefinger to enable manipulation of IV needle assembly 24. Wings 40, however, also can be rotated into a substantially coplanar disposition for taping to the skin of a patient.

IV needle assembly 24 further includes a tubular shield 42 that is telescoped over IV hub 26. Shield 42 is formed with transverse slots 44 that slidably receive wings 40. Thus, shield 42 can be slid from a proximal position, as shown in FIGS. 1A and 1B to a distal position. IV cannula 32 is exposed for use when shield 42 is in the proximal position shown in FIGS. 1A and 1B. However, IV cannula 32 is substantially surrounded by shield 42 when shield 42 is moved to the distal position. Additionally, slots 44 in shield 42 are configured to lockingly engage wings 40 when shield 42 is in the distal position to prevent or complicate a re-exposure of IV cannula 32. The shield illustrated in FIGS. 1A and 1B is one of many optional shield designs that can be incorporated into blood collection set 10. Other designs may provide wings mounted directly to the shield. Still other designs may provide a hinged shield mounted to IV hub 26. In still other designs, a shield may be entirely separate from IV needle assembly 24 or a shield may not be provided at all. Moreover, an unshielded set is also possible according to the invention.

Blood collection set 10 further includes a length of flexible plastic tubing 46. Tubing 46 includes opposite proximal and distal ends 48 and 50 and a passage extending between the ends. Distal end 50 of tubing 46 is securely mounted to proximal end 28 of IV hub 26 so that the passage through IV hub 26 communicates with the passage through tubing 46. A female luer fitting 52 is securely mounted to proximal end 48 of tubing 46.

Blood collection set 10 further includes a non-patient needle assembly 54, as shown in FIG. 2. Non-patient needle assembly 54 includes a non-patient hub 56 with a proximal end 58, a distal end 60 and a fluid passage 62 extending between the ends. Exterior surface regions of non-patient hub 56 substantially adjacent proximal end 58 define an array of external threads 64 configured for threaded engagement with the internal threads formed in mounting aperture 22 of collection tube holder 12. External surface regions of non-patient hub 56 adjacent distal end define a male luer taper 66 configured for mating with female luer fitting 52. Non-patient needle assembly 54 further includes a non-patient cannula 68 having a pointed proximal end 70, a distal end 72 and a lumen 74 extending between the ends. Distal end 72 of non-patient cannula 68 is mounted securely in passage 62 through non-patient hub 56 and aligns substantially with external threads 64 on non-patient hub 56. Non-patient needle assembly 54 further includes a multiple sample sleeve 76 mounted over non-patient cannula 68 and securely engaged with proximal end 58 of non-patient hub 56. Multiple sample sleeve 76 effectively functions as a valve that prevents a flow of fluid from non-patient cannula 68. However, multiple sample sleeve 76 can be pierced by pointed proximal end 70 of non-patient cannula 68 in response to forces generated by a stopper on an evacuated collection tube.

Blood collection set 10 is employed by folding wings 40 into face-to-face engagement with one another and gripping wings 40 between a thumb and forefinger. Any packaging cover that may be mounted over IV cannula 32 then is removed and discarded. Pointed distal end 36 of IV cannula 32 then is urged into a targeted blood vessel. The healthcare practitioner then may release the grip on wings 40, and if long term access to the blood vessel is required, wings 40 may be taped into face-to-face engagement with the skin of the patient. Blood collection set 10 includes a plurality of internal spaces that will initially be at ambient air pressure. These internal spaces include lumen 38 through IV cannula 32, the passage through IV hub 26, the passage through flexible tubing 46, passage 62 through non-patient hub 56 and lumen 74 through non-patient cannula 68. The venous or arterial access achieved with IV cannula 32 places these interior spaces of blood collection set 10 in communication with the pressure of the blood in the patient. Blood pressure exceeds the ambient air pressure. Accordingly, the pressure of air in the above-referenced internal spaces will increase, and blood will begin to flow into these internal spaces. As discussed above, prior art systems may reach equilibrium as the air pressure within the blood collection set increases in response to a reduction of volume caused by the inflow of blood. Hence, a portion of the internal spaces in the prior art system may remain filled with air at a pressure substantially equal to the venous or arterial pressure. Stated differently, a prior art system will include its original volume of air in the space between the proximal end of the non-patient needle and the blood that enters the blood collection set. This high-pressure air will escape into the first evacuated collection tube that is placed in communication with the non-patient needle. Hence, the first collection tube employed with prior art systems normally is a discard tube. With the subject invention, however, the communication of blood at venous or arterial pressure with the interior spaces of blood collection set 10 will urge air through a venting mechanism. Various embodiments of such venting mechanisms are described in detail below.

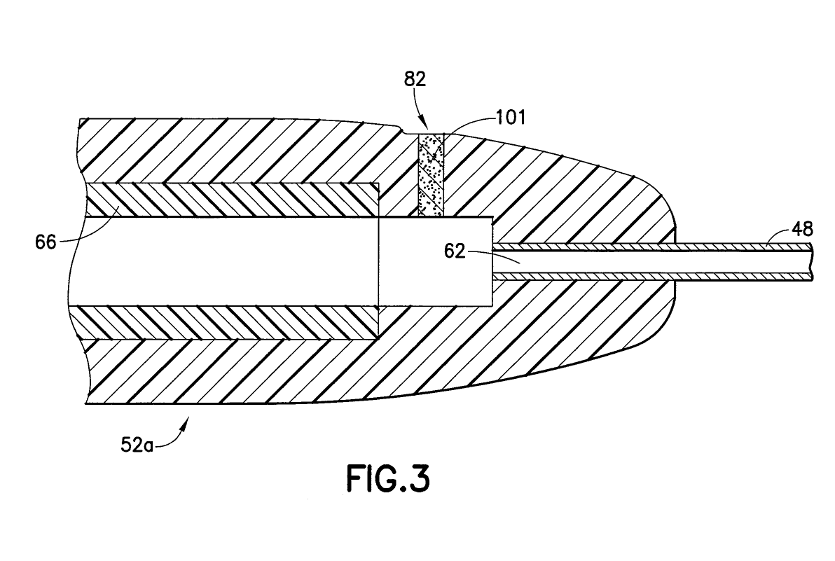

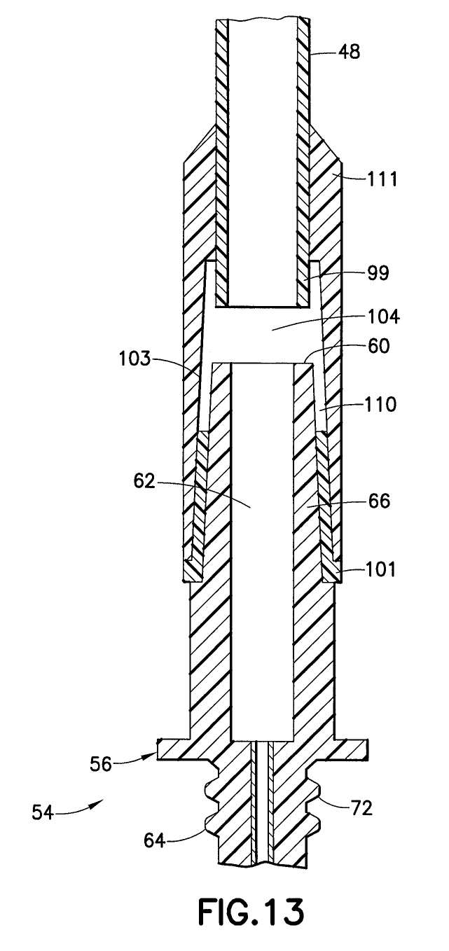

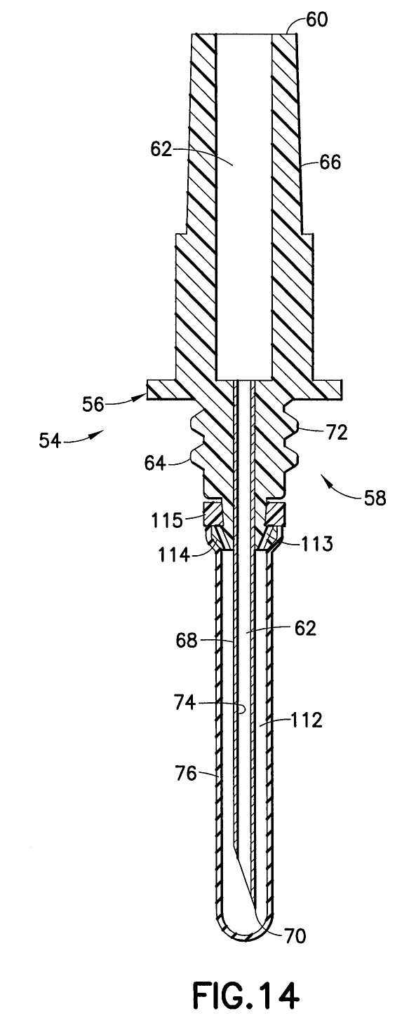

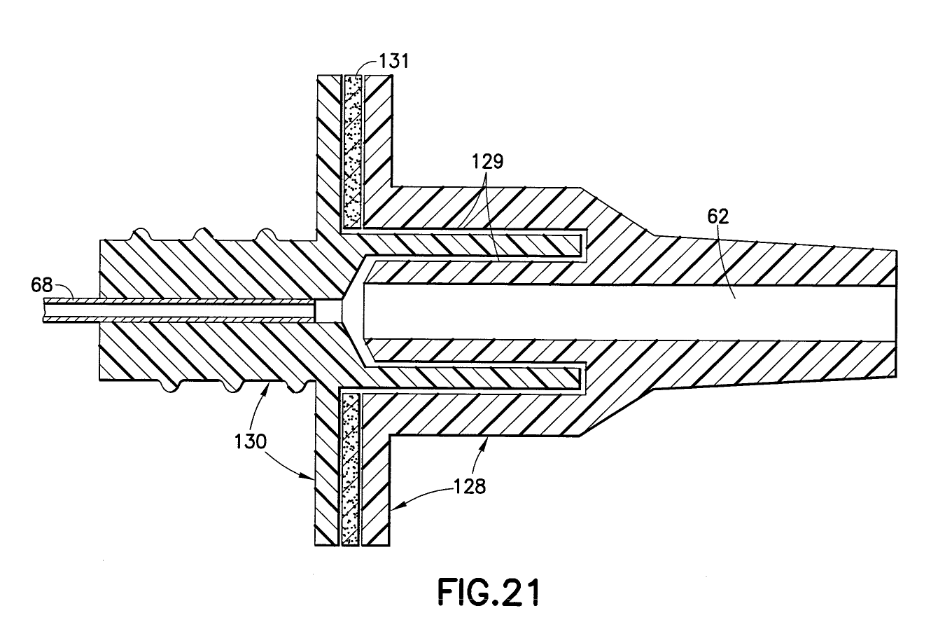

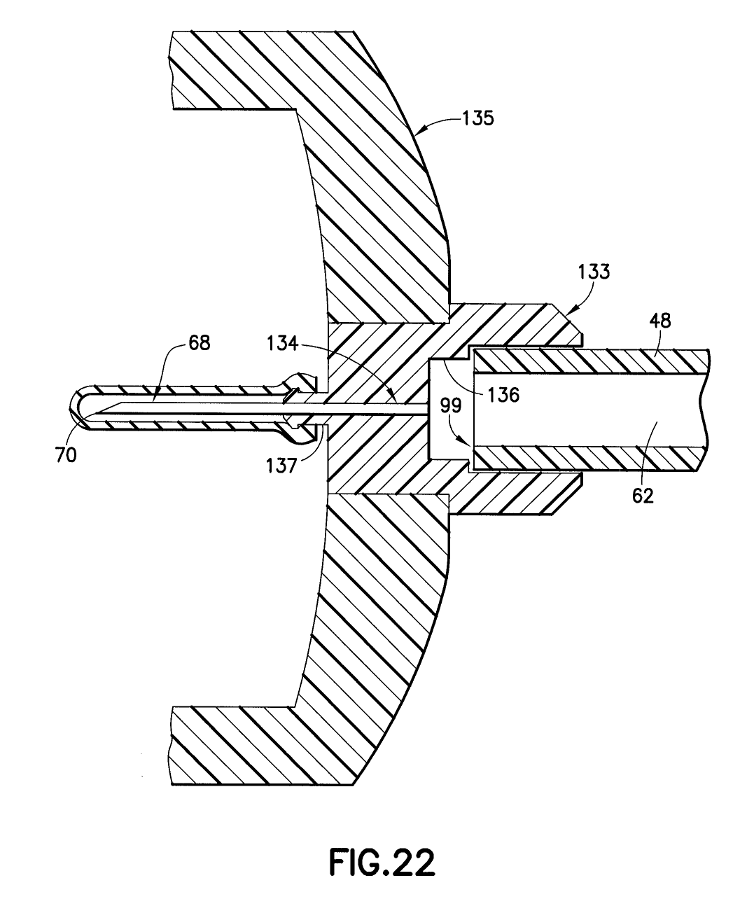

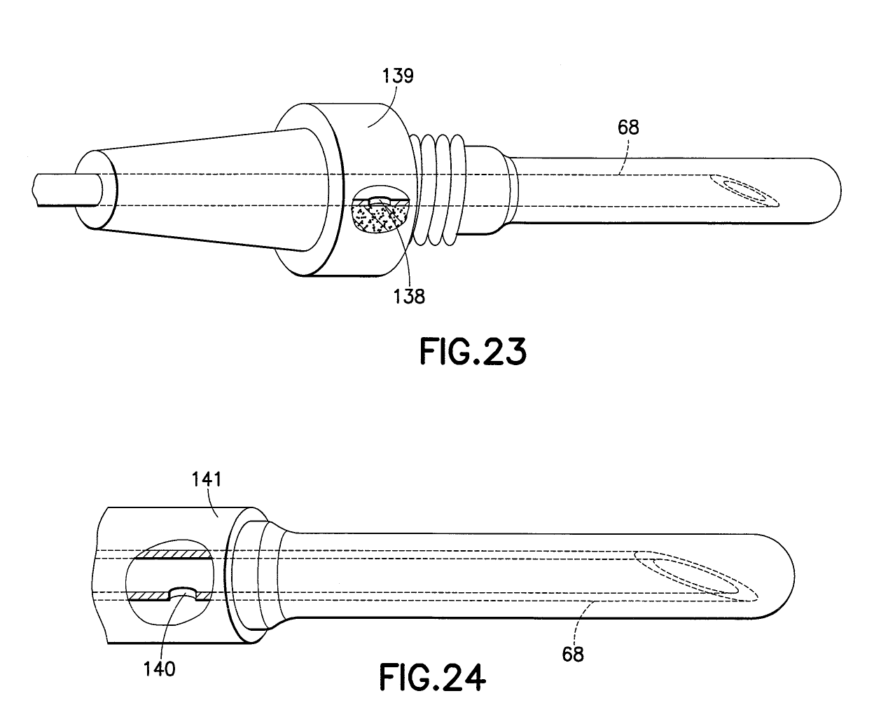

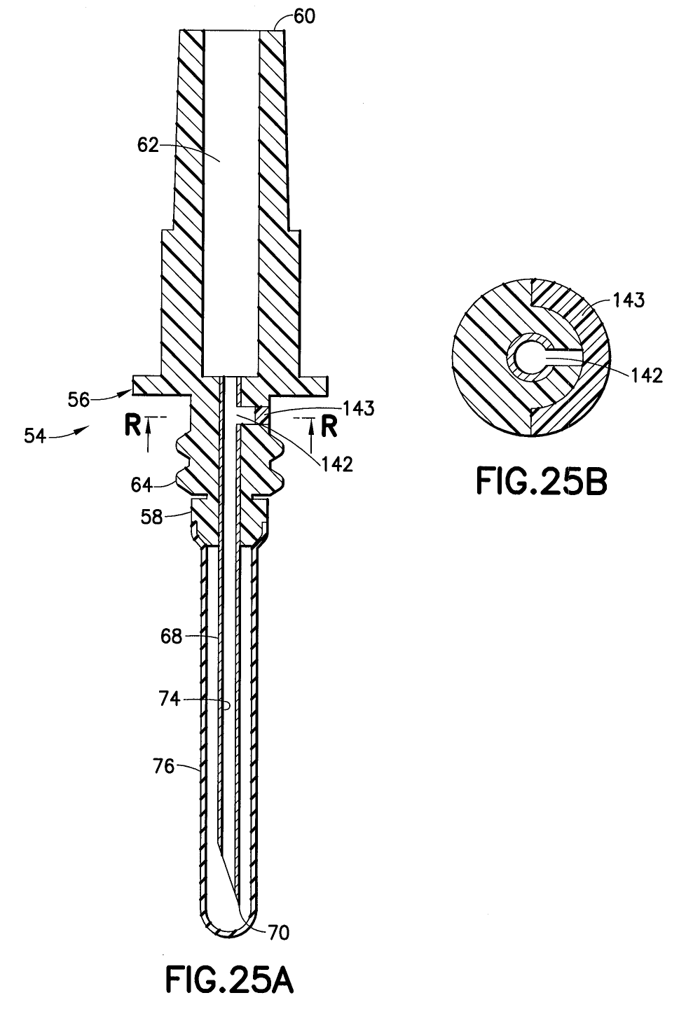

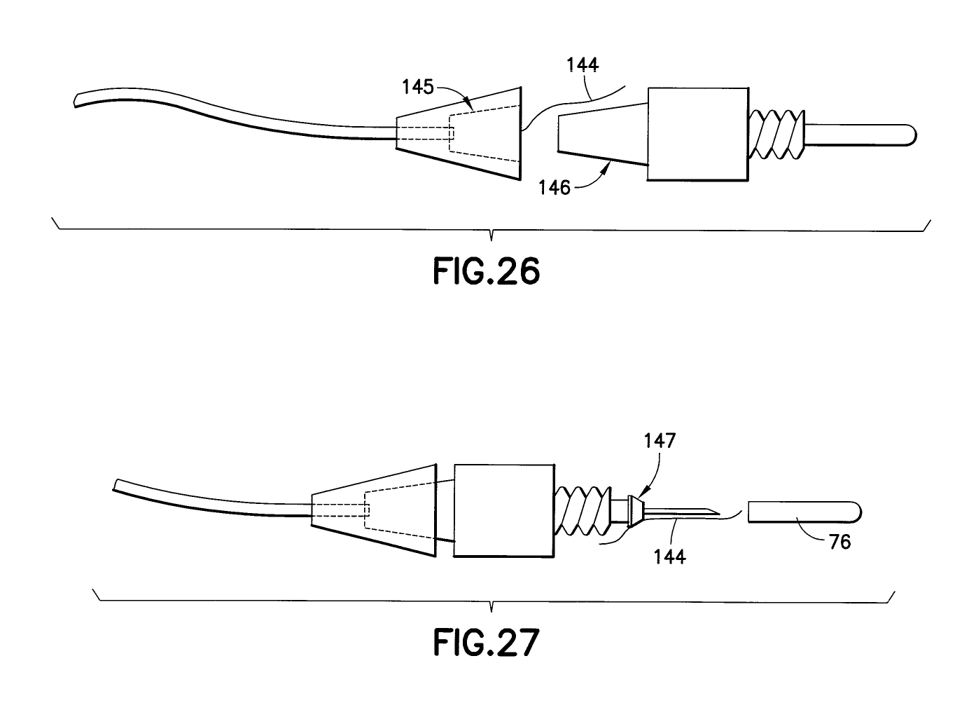

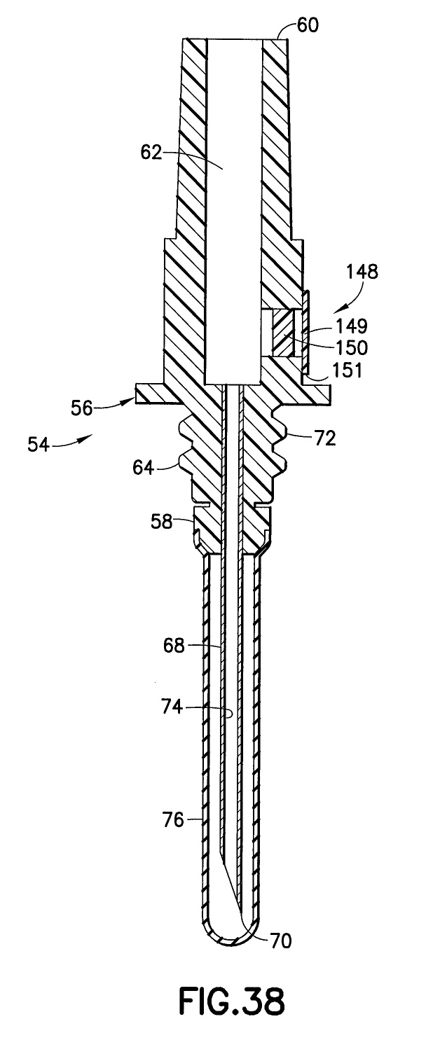

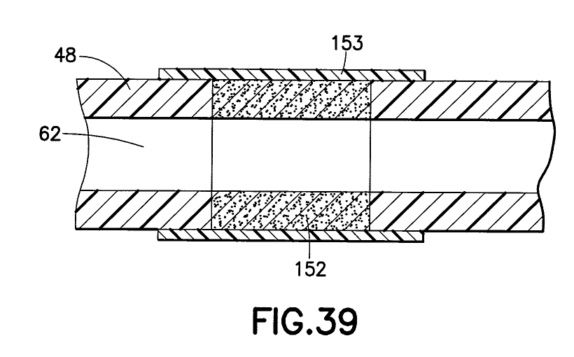

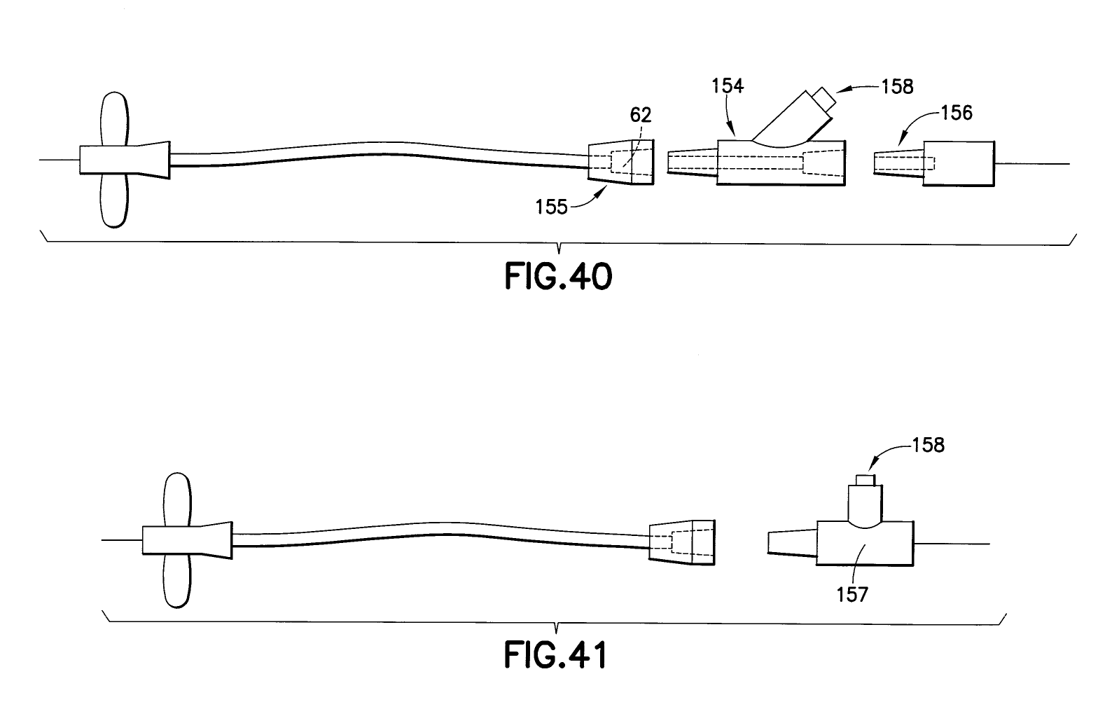

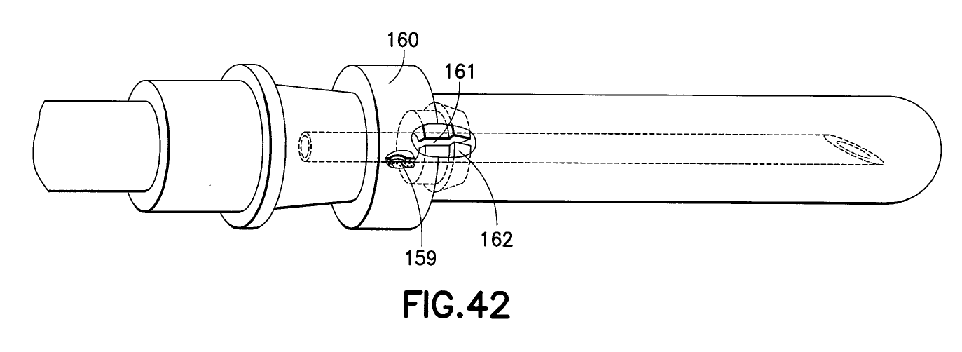

FIGS. 3-42 show various embodiments of the invention, including various configurations of venting mechanisms in blood collection tubing sets. In particular, FIGS. 3 to 12 reflect embodiments in which a venting mechanism is located in the female luer 52 portion. FIG. 13 reflects embodiments where the venting mechanism is located between the interface of the female luer 111 and the male luer taper 66. FIGS. 14-20 reflect embodiments where the venting mechanism is located beyond the proximal end 70 of the non-patient cannula in the non-patient hub 56. FIG. 21 reflects an embodiment where the venting mechanism is remote from the blood collection flow path and that communicates with it through a tortuous path in the non-patient hub. FIG. 22 reflects an embodiment where the venting mechanism is a unified non-patient hub 132. FIGS. 23 and 24 reflect embodiments where the venting mechanism is a hole in the non-patient cannula. FIG. 25 reflects an embodiment where the venting mechanism is located between male luer wall 125 and the threads 64 of the non-patient hub. FIGS. 26 to 37 reflect embodiments where the venting mechanism is a breathable cord. FIGS. 38 and 39 reflect embodiments where the venting mechanism is a one-way valve. FIGS. 40 and 41 reflect embodiments where the venting mechanism is a “Y” or “T” branching in the fluid passage into which the air is displaced. FIG. 42 reflects an embodiment of a combination of two venting mechanisms located in the non-patient hub.

FIG. 3 shows a venting mechanism that includes an aperture 82 extending radially from the interior of the of the luer 52a to the exterior. A venting plug 101 is located within at least a portion of the aperture 82. Aperture 82 thereby provides communication between the fluid passage 62 and the ambient surroundings allowing air to escape but preventing the outflow of blood or other fluids. Venting plug 101 (and like elements described herein) may be formed from any suitable material.

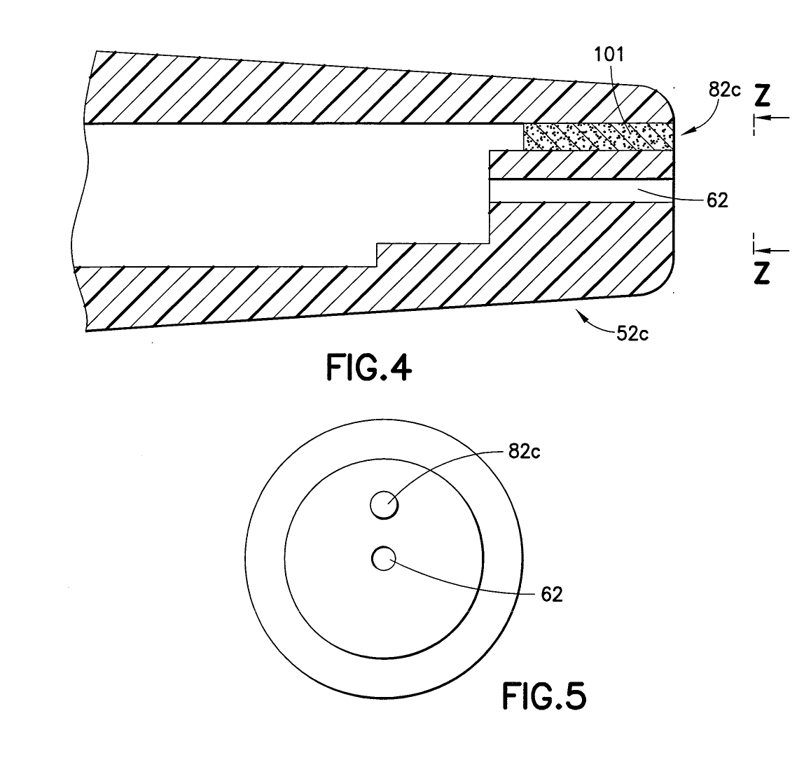

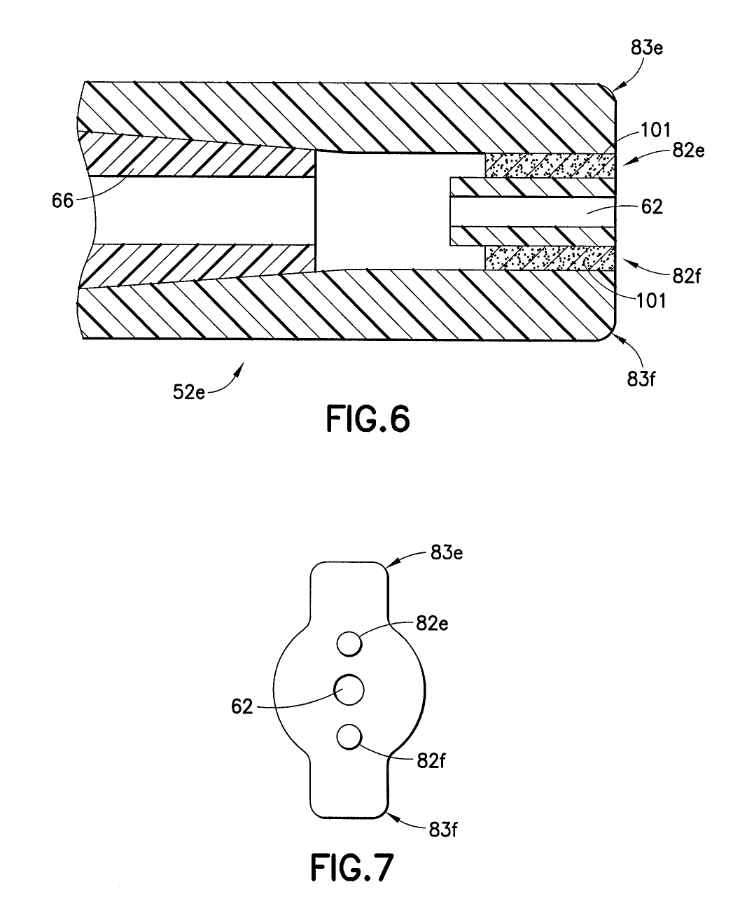

FIG. 4 shows a luer 52c, having an axially extending aperture 82c and a venting plug 101 located therein, the aperture running substantially, parallel to the fluid passage 62. As in the above embodiment, the aperture provides communication between passage 62 of non-patient hub 56 and the ambient surroundings. FIG. 5 shows a side view of FIG. 4 from the aspect of Z-Z. An alternative embodiment is shown in FIGS. 6 and 7 in which the venting mechanism comprises two axially extending apertures 82e and 82f each containing a venting plug 101. In addition, in this embodiment, the luer 52c contains two projections 83e and 83f in which the apertures are located. In other embodiments, one or more apertures may be located outside the projection. These projections also serve to facilitate the removal of the female luer 52e off the mating male luer 66. In alternative embodiments, there maybe any number of projections and any vent number of vent apertures. Each projection does not necessarily have a vent aperture.

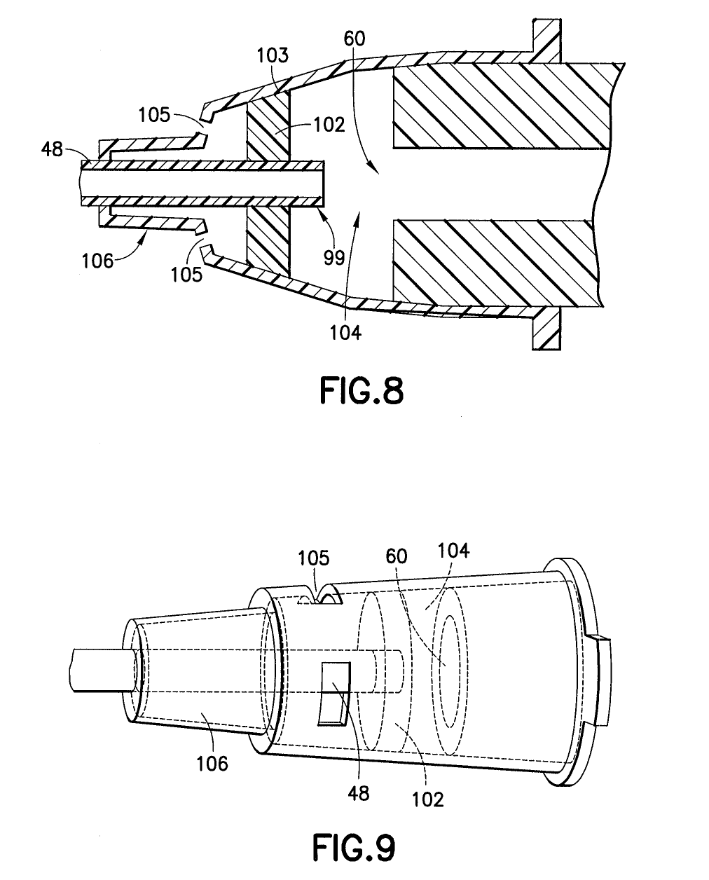

FIGS. 8 and 9 show another female luer venting mechanism in which a venting ring 102 is situated around the proximal end 99 of the flexible tube 48. The venting ring forms an interference fit with the inside female luer taper 103. A fluid chamber 104 is formed between the interface of the venting ring/proximal end of the tubing and the non-patient hub distal end 60 when the male luer taper is mated to the female luer, such that air can escape by passing through the venting ring and out of venting apertures 105 to the ambient surroundings. The apertures may be located anywhere in the body of the female luer. In FIGS. 8 and 9, the apertures are located in the distal end of the female luer 106. An alternative embodiment can be send in FIG. 10 in which the venting apertures are longitudinal venting windows 107 which abut the distal side of the venting ring 102.

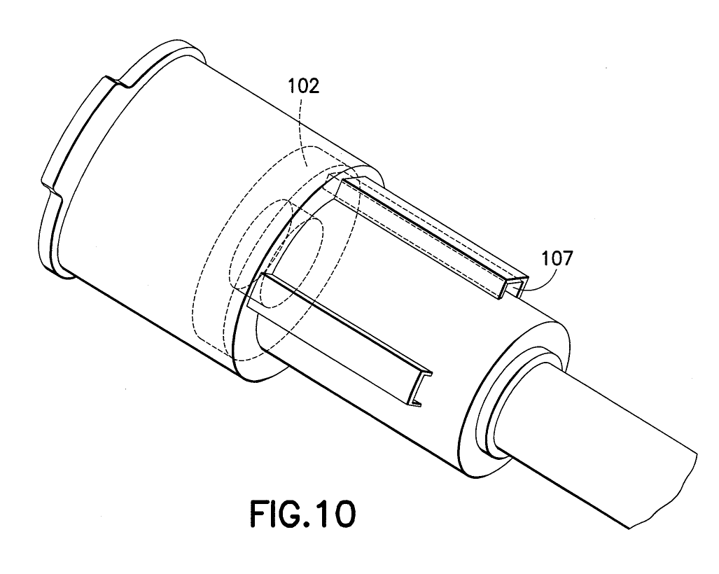

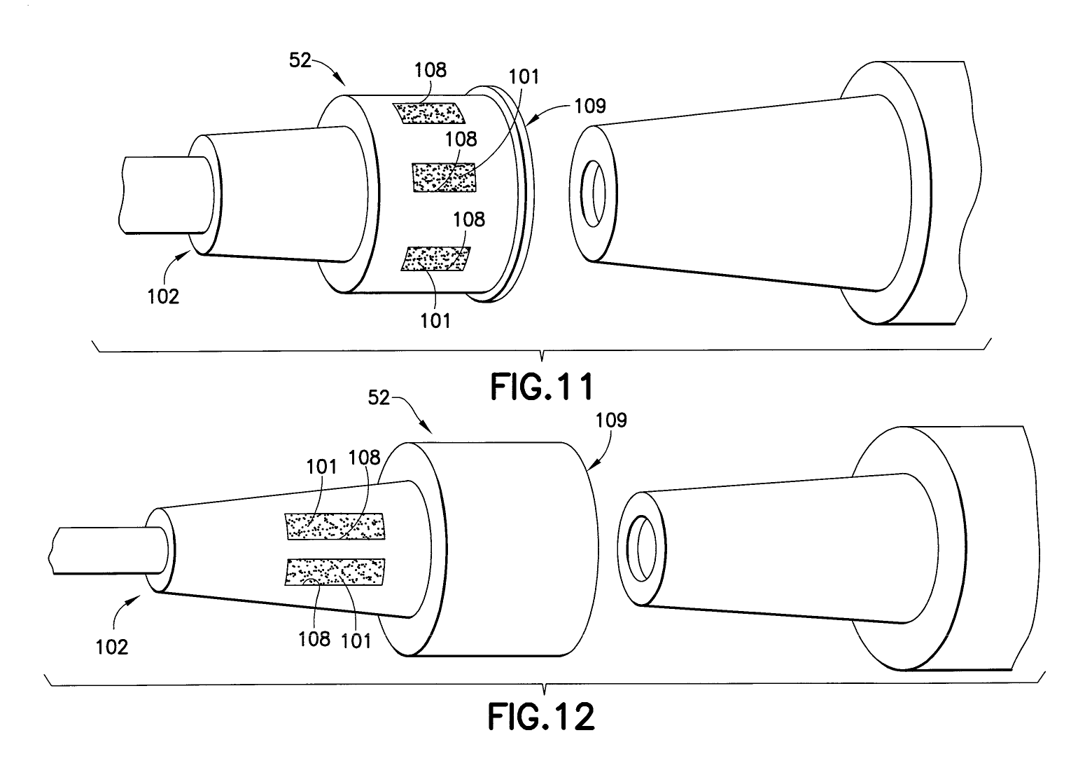

FIG. 11 shows a venting mechanism in which a series of transverse vent apertures 108, each containing a venting plug 101 are located in the body of the luer 52. In FIG. 11, the apertures are spaced equally around the circumference of the proximal end of the female luer fitting 109. Similarly FIG. 12 shows a series of transverse vent apertures 108, each containing a venting plug 101, that are equally spaced around the circumference of the distal end of the female luer fitting 52. In both cases a fluid chamber exists between the male luer taper and the female luer taper surfaces where air can flow out of the fluid passage and out of the vent mechanism.

FIG. 13 shows a venting mechanism that includes a venting plug 101 at a location between the interface of the female luer 111 and the male luer taper 66, which provides communication between passage 62 of non-patient hub 56 and the ambient surroundings. The venting plug 101 acts as a spacer and forms an interference fit with the inside female luer taper 103. A fluid chamber 104 is formed between the interface of the female luer taper/proximal end of the tubing 99 and the non-patient hub distal end 60 when the male luer taper is mated to female luer with the venting plug located in-between, such that air can escape by passing through the gap 110 between the male luer and female luer tapered surfaces and the venting plug 101. Note that in this embodiment, as in the other embodiments herein, the vent material could be a variety of materials or elements. Typically configurations for this embodiment may include, for example, a porous ring formed from a hydrophobic material or which has a hydrophobic surface, a porous ring that becomes sealed upon contact with blood using biological phenomena, a ring of swellable material, a textured surface, or a coating of a swellable or similar material.

Several embodiments involve a venting mechanism location beyond the proximal end 70 of the non-patient cannula. In the embodiment of FIG. 14, air flows from the fluid passage 62 and out of the non-patient cannula proximal end 70 where it further flows through the space 112 between needle exterior 68 and multiple sample sleeve 76 interior to the location of the vent 113, which consists of a passage through the non-patient barb 114 then through the venting plug 115 that permits an outflow of air, but prevents an outflow of blood or other fluids to the ambient surroundings.

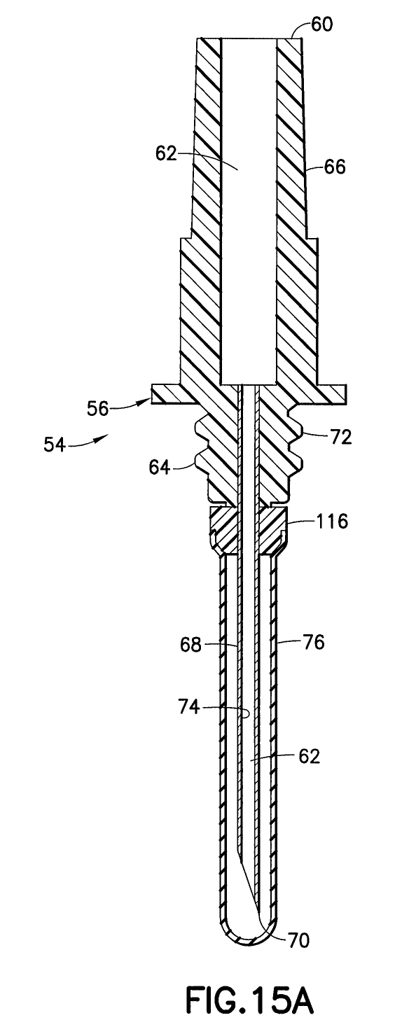

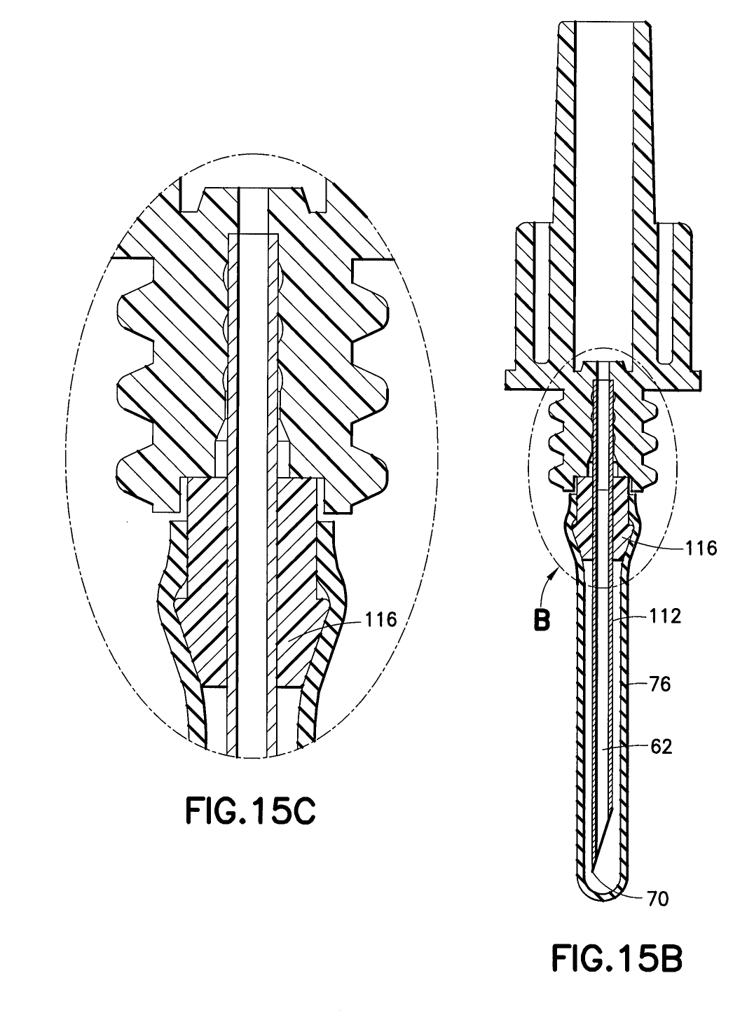

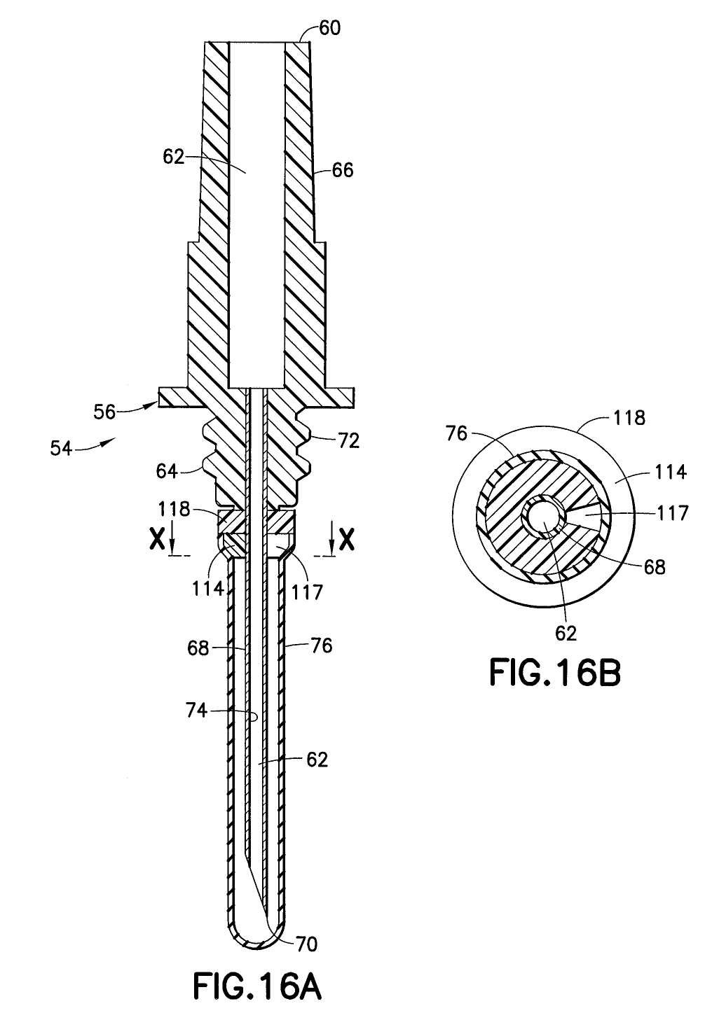

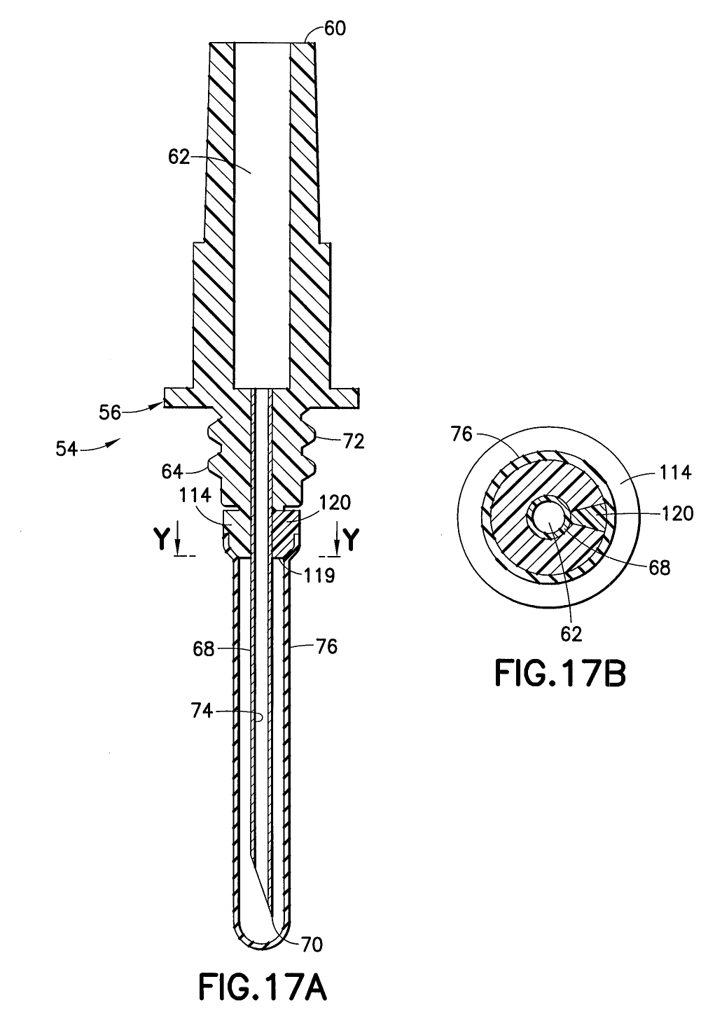

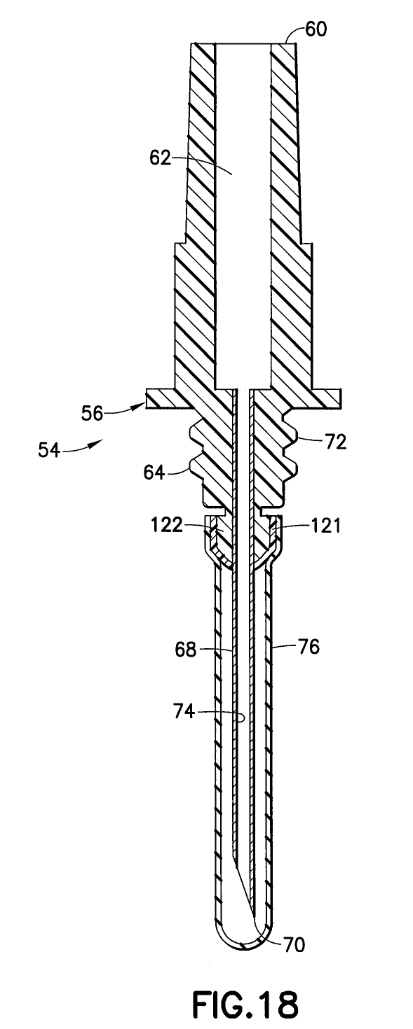

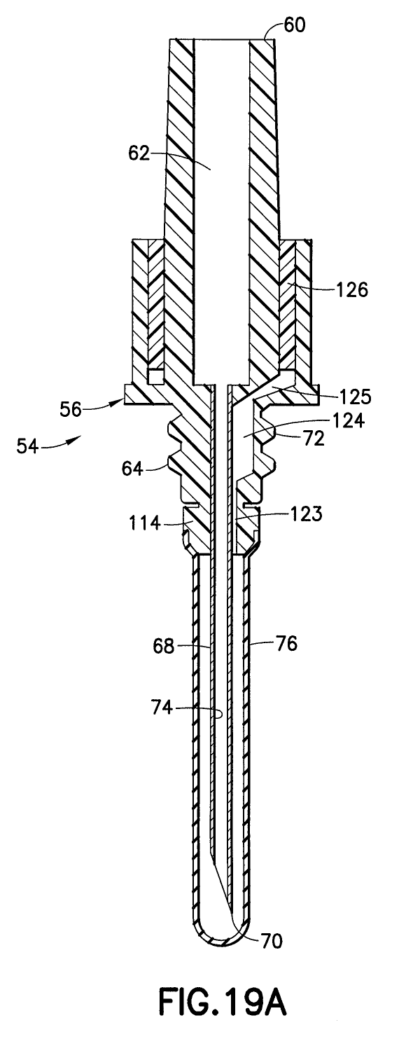

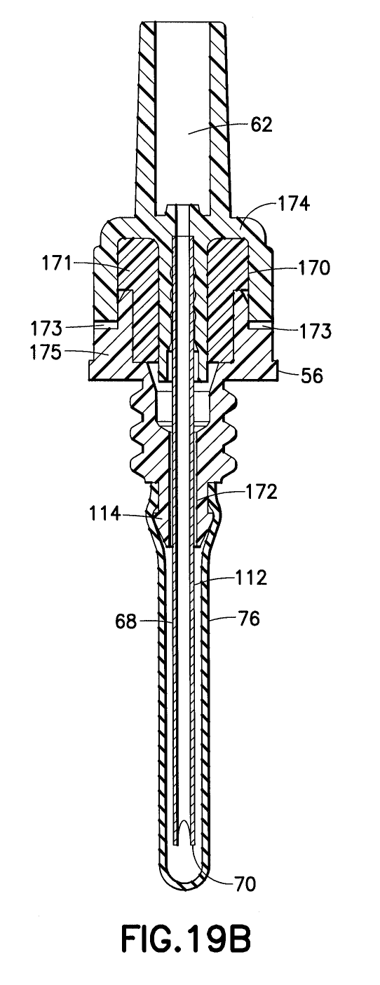

FIGS. 15A, 15B, 15C, 16A, 16B, 17A, 17B, 18, 19A and 19B demonstrate more embodiments where venting mechanisms are located beyond the proximal end 70 of the non-patient cannula. As seen in FIG. 14 air flows from the fluid passage 62 and out of the non-patient cannula proximal end 70 where it further flows through the space 112 between needle exterior 68 and multiple sample sleeve 76 interior to the location of the vent which is situated through or around or part of the non-patient barb. The embodiments in FIGS. 15A, 15B and 15C show a venting plug 116 that also functions as the non-patient barb. FIGS. 16A and 16B show an embodiment in which a slit 117 in the non-patient barb 114 allows air to escape to a venting disc 118. FIGS. 17A and 17B show an alternative embodiment in which a slit 119 in the non-patient barb 114 contains a venting plug 120. FIG. 18 shows a modified non-patient barb design 122 in which a venting sleeve 121 allows air to escape. FIG. 19A shows a further embodiment in which air escapes through a small channel 123 in the non-patient barb 114 into a thread reservoir 124 then through a channel in the male luer wall 125 into a cylindrical venting plug 126. FIG. 19B shows another embodiment in which the non-patient hub is made from 2 separate parts; the male luer 174 and the non-patient thread assembly 175. Air escapes through a small channel 172 inbetween the needle exterior 68 and the non-patient barb 114 and then through the male luer wall 56 into a reservoir 170 in the male luer that contains the vent media 171 and out to the surrounding atmosphere through a channel 173 at the interface between the male luer 174 and the non-patient thread assembly 175 of the non-patient hub. In each embodiment the vent media (and like elements described herein) permits an outflow of air, but prevents an outflow of blood or other fluids to the ambient surroundings.

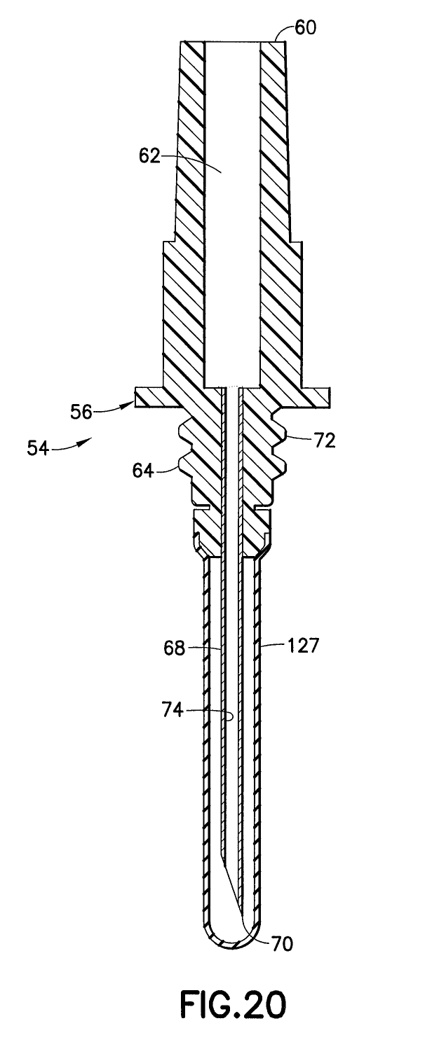

FIG. 20 shows another embodiment of the venting mechanism location beyond the proximal end 70 of the non-patient cannula. According to this embodiment, air is vented through the material of the multiple sample sleeve 127 itself, which functions as the vent media that prevents a flow of fluid from non-patient cannula 68. Multiple sample sleeve 127 maintains its normal function of being pierced by pointed proximal end 70 of non-patient cannula 68 in response to forces generated by a stopper on an evacuated collection tube. It is possible to make a multiple sample sleeve 127 that also functions as a vent by forming the sleeve from a porous hydrophobic material, such as those disclosed above.

FIG. 21 shows a venting mechanism that is remote from the blood collection flow path and that communicates with it through a tortuous path, thereby preventing contamination of blood in the fluid passage by contact with the vent media. Air flows from the fluid passage 62 within the male luer 128 into a tortuous air pathway 129 that is formed by the male luer 128 and the non-patient cannula hub 130. Air then passes from the tortuous air pathway 129 to the vent media, which is an annular venting ring 131 and into the ambient surroundings.

FIG. 22 shows a venting mechanism that is a unified non-patient hub 132, at least a portion of which is made from a porous material. This porous portion of the hub 132 itself thus acts as the vent media. The proximal end 99 of the flexible tubing 48 is bonded to the distal end of the unified non-patient hub 133. The non-patient cannula 68 is bonded to the proximal end of the unified non-patient hub 134. And because of the nature of the unified non-patient hub 132, the holder will typically be pre-attached by bonding, rather than by providing threads on the hub 132. However threads can be provided if desired. Bonding of the tube holder 135 to the unified non-patient hub 134 may be accomplished by solvent, welding, heat, pressure or and other convenient means or combination thereof. Air flows through the fluid passage 62 and vents to the ambient surroundings by passing through the unified non-patient hub 132. An alternative embodiment incorporates an impermeable spot coat to the inside surface wall 136 of the unified non-patient hub 132 that is part of the fluid passage 62. This results in air only being able to vent through the hub at the non-patient barb 137 beyond the proximal end 70 of the non-patient cannula.

In the embodiment of FIGS. 23 and 24, the venting mechanism is an opening in the non-patient cannula, surrounded by the vent media, which is shown as a vent plug. FIG. 23 shows a hole 138 in the non-patient cannula 68. Air flows from the fluid passage 62 and out through the hole 138 into a porous venting ring 139 then into the ambient surroundings. In FIG. 24, the hole 140 in the non-patient cannula 68 is situated at the non-patient barb 141, which also acts as a venting ring. This allows air to escape through the hole 140 that was in the fluid passage between the IV needle assembly and through the non-patient barb 141 for air in a further proximal location down the fluid passage stream after said hole 140

FIGS. 25A and 25B show a venting mechanism that is located between male luer wall 125 and the threads 64 of the non-patient hub. Air flows from the fluid passage 62 into the vent hole 142 and then through the porous C clamp vent plug 143 to the ambient surroundings. As discussed above the vent plug material in this embodiment typically has an elastic property and shape such that spring energy holds the vent material onto the device. In this embodiment the distortion of the C-shaped vent plug shape is required for the vent plug to stretch over the receiving structure on the hub. Once the vent plug is placed over the receiving structure, it is released and fully maintained in place using its own resiliency and in absence of bonding materials such as epoxies, which could be disadvantageously absorbed into the vent plug. The vent mechanism of this embodiment could alternatively involve first compressing the vent material, placing the vent material into the opening, and releasing the vent material to expand into the opening.

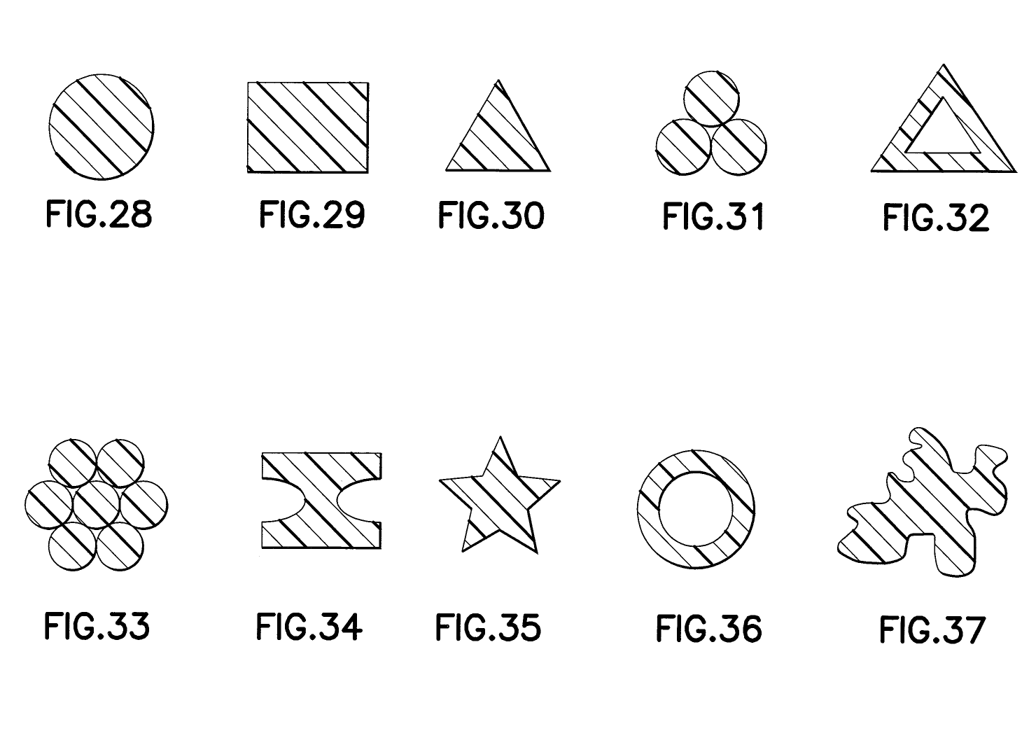

FIGS. 26 and 27 show the venting mechanism of a breathable venting cord at two different locations along the fluid passage, though venting using this mechanism may be accomplished by locating the vent media (e.g., placing, coating, or treating) between any one or multiple sealing surfaces along the fluid passage. FIG. 26 shows the cord 144 located between the sealing surfaces of the female 145 and male 146 luer tapers. The presence of the cord in the sealing surface allows air to escape from the fluid passage 62 but prevents leakage of a fluid through either the absorbent nature of the cord material and/or the very small size of the channel created by the cord. FIG. 27 shows the cord located between the sealing surfaces of the multiple sample sleeve 76 and non patient hub barb 147. FIGS. 28 to 37 show cross-sections of suitable breathable cords. Other shapes, or combinations of such profiles, may also be used. Cords may be extruded or woven, for example and the application of a hydrophobic coating such as wax maybe advantageous.

In a further embodiment, the venting mechanism utilizes a one way valve located somewhere along the fluid passage. The valve allows air to escape but shuts closed when vacuum is applied thus, when an evacuated collection tube is applied at the needle tip, the tube draws blood from the fluid passage but not air. FIGS. 38 and 39 show examples of a one-way valve. The venting mechanism may be at any location or locations along the fluid passage 62, but is typically at the proximal end of the non-patient hub 56. The valve 148 itself may be a thin flap such as plastic film 149 covering the vent, a deformable seal such as a rubber or plastic duckbill valve, a deformable wrap over the vent, or any other means or combination of these. The valve 148 may be proximal or distal with respect to the vent. In the embodiment shown in FIG. 38 the thin plastic film valve 149 is attached to the non-patient hub 56 along one sealed edge of the film 151, so that on the initial venous puncture, air is pushed out of the fluid passage 62 under venous pressure through the porous vent plug 150 and out from underneath the unsecured edges of the plastic film 149. However when a vacuum is applied to the fluid passage 62 (via the attachment of a blood collection tube) the thin plastic film valve 149 is pulled tight against the porous vent plug 150 thereby sealing the vent and preventing air from reentering the fluid passage. This embodiment may thus provide a primary or back-up feature to prevent air from re-entering the system after venting occurs. An alternate embodiment is shown in FIG. 39, in which the blood flows from a length of conventional flexible tubing 48 into a venting mechanism that consists of a length of porous tubing 152, which is loosely wrapped around its outer surface in a length of a non-porous flexible film 153. This wrap 153 is sufficiently loose to allow air to escape on initial venous puncture such that under a vacuum, it is pulled tight and seals the outer surface of the length of porous tubing 152.

FIGS. 40 and 41 show a venting mechanism using a “Y” or “T” branching in the fluid passage into which the air is displaced. The branching may be at any location or locations along the fluid passage, but is typically at the proximal end such as at the non-patient hub. FIG. 40 shows a “Y” Branch Vent 154 in the form of a separate component added into the fluid passage 62 such as in between the female 155 and male luer 156 fittings. FIG. 41 shows a “T” Branch Vent 157, which is an integral part of the non-patient hub, thus reducing the number of components. In each embodiment the vent plug 158 allows the air to escape but prevents leakage of blood. The use of a “Y” or “T” Branch Vent may be accomplished with any convenient terminal shapes at the interfaces of each component. The embodiments exemplified previously are shown with luer tapered fittings but other interface designs such as direct connection to the flexible tubing may also be applied. Although typical applications use a three port “Y” or “T” branch vent the use of a multiple port branch vent system is also possible.

As will be apparent to one skilled in the art, it is possible to combine one or more vent mechanisms in a single device, or put identical vent mechanisms at more than one location in a device. Moreover, it is possible to use any of a variety of vent plugs in the vent mechanisms of the invention. In addition, vent mechanisms herein may be applicable in a variety of devices other than blood collection sets.

For example, FIG. 42 shows a combination of two venting mechanisms. Air flows from the fluid passage 62 and out through the hole 159 in the non-patient cannula 68 into a porous venting ring 160 then into the ambient surroundings. A second vent path exists past beyond the proximal end 70 of the non-patient cannula. Air that is proximal to the hole 159 location flows from the fluid passage 62 and out of the non-patient cannula proximal end 70 where it further flows through the space 112 between needle exterior 68 and multiple sample sleeve 76 interior to the slit 161 in the non-patient barb 162 also allows air to escape to the porous venting ring 160.

Vent media, as used herein, can include, for example, either or a combination of:
a porous plug formed from a matrix or carrier material, typically hydrophobic, that is coated with, impregnated with, or otherwise contains a hydrophilic material that swells on contact with aqueous or water containing substances. This swellable nature thereby provides the sealing function in the vent upon contact with blood;
an air vent provided through a matte finish, micro-sized channels, laser drilled holes, tortuous path, or a vent provided between sealing surfaces, e.g., in a cord in which the holes, gaps or channels are large enough to permit airflow but small enough to prevent blood leakage;
a porous plug that becomes sealed upon contact with blood using biological phenomena, e.g., by clotting and/or cell agglutination that blocks the vent;
a superabsorbant material to seal the vent by swelling on contact with a aqueous fluid; or
a one-way valve, e.g., a thin flap such as plastic film covering a vent, a deformable seal such as a rubber or plastic duckbill valve, or a deformable wrap over a vent.

Typically, a porous plug is formed from a hydrophobic material, such as high-density polyethylene (HDPE), which is coated with, impregnated with, or otherwise contains a hydrophilic material such as carboxymethylcellulose (CMC) or a polyacrylate. Alternative hydrophobic materials include but are not limited to polytetrafluoroethylene (PTFE), ultra-high molecular weight polyethylene (UHMWPE), Nylon 6, polypropylene (PP), polyvinylidine fluoride (PVDF) or polyethersulfone (PES).

An embodiment of the vent media consists of micro-sized holes formed in an exterior wall. The holes are large enough to permit airflow but small enough to prevent blood leakage. The vent holes may be any number including a single hole although multiple holes are typical for a more reliable function. The holes may be laser-drilled, meaning that they may be burned through the wall or substrate using one or more laser beams. The substrate may be any convenient material although thin plastic or plastic film is typical. The vent mechanism may include a one-way valve as previously described. The vent mechanism may be located at any convenient space along the fluid passage in the flexible tubing, luer or non-patient hub or in an added component although location at the proximal end is typical to provide flash along the full length of the tubing.

A porous plug that becomes sealed upon contact with blood using biological phenomena may use, for example, a porous material such as a sintered plastic, ceramic or metal, or a breathable cord, or by locating the biological agent in small holes or spaces between parts. The vent may be of any convenient shape. The venting may be at any location or locations along the fluid passage, but is preferably at the proximal end such as at the hub near the collection device. The vent is typically made from, contains, is adjacent to, or works in collaboration with, a stimulant that interacts with blood to promote clotting and/or cell agglutination such that the clot and/or clumped cells block ongoing flow of blood through the vent. An example a clotting stimulant is silica or crushed glass, or fiberglass. An example of an agglutinizing agent is lectin. An example of a platelet activator is collagen or thrombin. A neutralizer for anti-coagulant such as protomine sulfate may be included. The biological stimulant may be applied using any convenient process including as a powder, a solution, a suspension, a slurry, or any other form. It may be dried or lyophilized.

What is claimed is:

1. A device for drawing fluid from a lumen, comprising:
a central body having an outer wall and an inner fluid passage;
a front cannula incorporated into a butterfly needle housing, said front cannula communicating with the inner fluid passage at a first end of the central body;
a rear cannula communicating with the inner fluid passage at a second end of the central body;
a flexible sleeve surrounding at least a tip portion of the rear cannula; and
a means for venting air from the flexible sleeve, said means for venting being disposed between the rear cannula and the flexible sleeve, wherein the means for venting air includes a porous member, a porous collar, a porous insert, or a porous spacer, and wherein the means for venting air is outside of and adjacent to the rear portion of the central body.

2. The device of claim 1 further comprising means for observing fluid within the device.

3. The device of claim 2 wherein the means for venting air includes a base portion spaced from the central body.

4. The device of claim 3 further comprising a porous spacer disposed between the base portion and the central body.

5. The device of claim 4 wherein the porous spacer comprises absorbent material.

6. The device of claim 2 wherein the means for observing comprises a transparent or translucent member disposed between the front cannula and the central body.

7. The device of claim 6 wherein the transparent or translucent member comprises a flexible tube.

8. The device of claim 2 wherein the means for venting air comprises:
a non-porous member contacting a base portion of the rear cannula; and
a porous spacer disposed between the non-porous member and the central body.

9. The device of claim 8 wherein the non-porous member includes one or more venting features.

10. The device of claim 2 wherein the front cannula is connected to the central body by a flexible or semi-rigid tube.

11. The device of claim 10 further comprising a means for controlling blood flow through the front cannula.

12. The device of claim 2 further comprising a guide tube surrounding the rear cannula and connected to the central body.

13. The device of claim 2 wherein the means for venting air includes a base portion contacting the central body.

14. The device of claim 2 wherein the means for venting air is a porous member that includes a tapered portion and an annular recess adjacent the tapered portion.

15. The device of claim 2 wherein the means for observing comprises at least a portion of the central body that is transparent or translucent.

16. The device of claim 7 wherein the transparent or translucent member comprises at least a portion of a Luer hub.

17. The device of claim 2 wherein the means for venting air is substantially porous to air and substantially non-porous to blood.

18. The device of claim 2 wherein the means for venting air comprises:
a first member adapted to vent air; and
a second member substantially non-porous to blood.

19. The device of claim 2 wherein the means for venting air comprises:
a first member adapted to vent air; and
a second member adapted to absorb blood.

20. The device of claim 2 wherein the means for observing is adapted to magnify an image of the observed fluid.

21. The device of claim 2 wherein the means for venting air is substantially porous for gas constituents less than about 5 microns in size, and substantially non-porous for liquid constituents about 5 microns or greater in size.

22. The device of claim 2 further comprising a means for controlling blood flow through the device.

23. The device of claim 2 wherein the means for observing comprises a transparent or translucent portion of the butterfly needle housing.

24. The device of claim 1 wherein the means for venting air does not contact the central body.

25. A device for drawing fluid from a lumen, comprising:
a central body having an outer wall, a rear portion and an inner fluid passage;
a front cannula communicating with the inner fluid passage;
a rear cannula communicating with the inner fluid passage and extending from the rear portion of the central body;
a sleeve surrounding at least a tip portion of the rear cannula;
a means for venting air disposed between the sleeve and the rear cannula, wherein the means for venting air includes a porous member, a porous cannula, a porous insert, or a porous spacer, and wherein the means for venting air is outside of and adjacent to the rear portion of the central body; and
a transparent or translucent member disposed between the front cannula and the central body.

26. The device of claim 25 wherein the transparent or translucent member comprises a flexible tube.

27. The device of claim 25 wherein the transparent or translucent member comprises at least a portion of a Luer hub.
